(19) United States
(12) Patent Application Publication
(10) Pub. No.: US 2010/0256206 A1
(43) Pub. Date: Oct. 7, 2010

(54) CRYSTALLINE FORM B OF OLMESARTAN MEDOXOMIL

(76) Inventor: Axel Becker, Hertfordshire (GB)

Correspondence Address: FULBRIGHT & JAWORSKI L.L.P.;Attn: MN IP Docket, 600 Congress Avenue, Suite 2400, Austin, TX, 78701, US

(21) Appl. No.: 12/602,948

(22) PCT Filed: Jun. 5, 2008
(86) PCT No.: PCT/GB08/50414

§ 371 Date: Jun. 2, 2010

(30) Foreign Application Priority Data
Jun. 5, 2007 (GB) 0710680.0

(51) Int. Cl. A61K 31/4178; C07D 407/14; A61P 9/12

(52) U.S. Cl. 514/382; 548/253

(57) ABSTRACT

The present invention is directed towards a novel crystalline form of olmesartan medoxomil, to methods for preparing the compound, to compositions comprising the compound, and to the use of said compound and compositions for the treatment or prevention of an angiotensin II receptor mediated disorder, in particular hypertension.

### CROSS-REFERENCE TO RELATED APPLICATION(S)

This application is a Section 371 National Stage Application of International No. PCT/GB2008/050414, filed 5 Jun. 2008 and published as WO 2008/149155 A1 on 11 Dec. 2008, which claims priority from the GB Patent Application No. 0710680.0, filed 5 Jun. 2007, the contents of which are incorporated herein in their entirety for all purposes.

### FIELD OF THE INVENTION

The present invention is directed towards a novel crystalline form of olmesartan medoxomil, to methods for preparing the compound, to compositions comprising the compound, and to the use of said compound and compositions for the treatment or prevention of an angiotensin II receptor mediated disorder, in particular hypertension.

### BACKGROUND OF THE INVENTION

Olmesartan medoxomil is described chemically as 2,3-dihydroxy-2-butenyl 4-(1-hydroxy-1-methylethyl)-2-propyl-1-[p-(o-tetrazol-5-yl-phenyl)benzyl]imidazole-5-carboxylate cyclic 2,3-carbonate and has the structural formula (I):

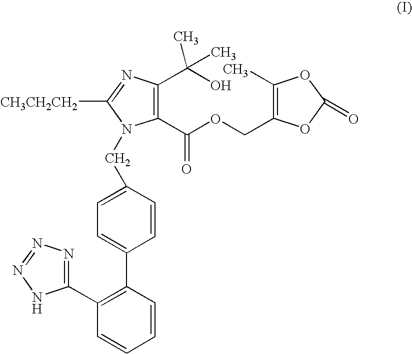

Olmesartan medoxomil is an anti-hypertensive pro-drug ester that is hydrolyzed to olmesartan during absorption from the gastrointestinal tract. It is a selective AT1 subtype angiotensin II receptor antagonist and blocks the vasoconstrictor effects of angiotensin II by selectively blocking the binding of angiotensin II to the AT1 receptor in vascular smooth muscle. Olmesartan medoxomil is indicated for the treatment of hypertension and is commercially sold under the trade name Benicar®.

EP 0503785 describes olmesartan medoxomil and discloses in example 61(b) a process for its preparation. The disclosed process results in a crystalline form characterized in The Annual Report of Sankyo Research Laboratories, vol. 55, 2003.

U.S. Patent Publication No. 2006/0281800 discloses form G olmesartan medoxomil as another crystalline form for use by the skilled person. However, performance data, for example, the results of stability or solubility testing, are not included in the disclosure.

Olmesartan medoxomil has a very low aqueous solubility. This can be problematic when developing pharmaceutical products, as solubility of the active pharmaceutical ingredient (API) is a key parameter to be considered. Prior art solutions to the problem of APIs with low aqueous solubility in general include the development of crystalline forms and amorphous forms having increased dissolution profiles.

Polymorphism is the occurrence of different crystalline and amorphous forms of a single compound and it is a property of some compounds and complexes. Polymorphs are distinct solids sharing the same molecular formula, yet each polymorph may have distinct physical properties. Therefore a single compound may give rise to a variety of polymorphic forms where each form has different and distinct physical properties, such as different solubility profiles, different melting point temperatures and/or different X-ray diffraction peaks. Since the solubility of each polymorph may vary, identifying the existence of pharmaceutical polymorphs is essential for providing pharmaceuticals with predicable solubility profiles. Polymorphic forms of a compound can be distinguished in a laboratory by X-ray diffraction spectroscopy and by other methods such as infrared spectrometry. Additionally, polymorphic forms of the same drug substance or active pharmaceutical ingredient can be administered by themselves or formulated as a drug product (also known as the final or finished dosage form), and are well known in the pharmaceutical art to affect, for example, the solubility, stability, flowability, tractability and compressibility of the drug substance and the safety and efficacy of drug products.

The discovery of new polymorphic forms of a pharmaceutically useful compound provides a new opportunity to improve the performance characteristics of a pharmaceutical product. It also adds to the material that a formulation scientist has available for designing, for example, a pharmaceutical dosage form of a drug with a targeted release profile or other desired characteristic.

### SUMMARY OF THE INVENTION

Due to the low aqueous solubility of olmesartan medoxomil, there is a need for alternative forms of this compound potentially having increased solubility for use in the development of pharmaceutical products. There is also a need for stable crystalline forms of olmesartan medoxomil suitable for pharmaceutical development.

It has now been surprisingly found that a new polymorphic crystalline form of olmesartan medoxomil exists.

The present invention provides a novel crystalline form B olmesartan medoxomil, processes for preparing said compound, and pharmaceutical formulations comprising said compound according to the invention.

Accordingly, a first aspect of the present invention provides crystalline form B olmesartan medoxomil.

In a second aspect there is provided crystalline form B olmesartan medoxomil having at least one of the characteristic XRPD peaks with 2θ values at 9.7°, 12.1°, 13.5°, 15.1°, 17.4°, 18.1°, and 21.2°±0.2°. Of course, it will be understood by the skilled person that the crystalline form B olmesartan medoxomil may in certain embodiments comprise any of the characteristic XRPD peaks, or in alternative embodiments may comprise any 7, 6, 5, 4, 3 or 2 of the characteristic XRPD peaks of the second aspect.

In a third aspect there is provided crystalline form B olmesartan medoxomil having at least five (preferably at least six, seven, eight, nine, ten, fifteen, twenty, twenty-five, thirty or thirty-five) of the characteristic XRPD peaks with 2θ values and d values at about:

| Angle | d value |
|---|---|
| 2-Theta ° | Angstrom |
| 7.4 | 11.89 |
| 8.5 | 10.44 |
| 9.2 | 9.62 |
| 9.7 | 9.08 |
| 10.8 | 8.20 |
| 11.2 | 7.89 |
| 11.8 | 7.51 |
| 12.1 | 7.28 |
| 12.9 | 6.88 |
| 13.5 | 6.56 |
| 14.7 | 6.02 |
| 15.1 | 5.88 |
| 16.0 | 5.54 |
| 16.8 | 5.28 |
| 17.4 | 5.08 |
| 18.1 | 4.91 |
| 18.8 | 4.72 |
| 19.6 | 4.53 |
| 19.9 | 4.47 |
| 20.8 | 4.26 |
| 21.2 | 4.19 |
| 21.5 | 4.12 |
| 22.1 | 4.03 |
| 22.3 | 3.98 |
| 22.8 | 3.89 |
| 23.6 | 3.77 |
| 24.3 | 3.66 |
| 24.9 | 3.57 |
| 25.4 | 3.50 |
| 25.7 | 3.46 |
| 26.3 | 3.38 |
| 27.7 | 3.21 |
| 28.1 | 3.17 |
| 29.3 | 3.04 |
| 29.8 | 3.00 |

In a fourth aspect according to the invention there is provided crystalline form B olmesartan medoxomil characterized by an X-ray powder diffraction pattern substantially as shown in FIG. 1.

A fifth aspect of the present invention provides crystalline form B olmesartan medoxomil characterized by a differential scanning calorimetry thermogram with an endothermic peak at about 181° C. The fifth aspect also provides crystalline form B olmesartan medoxomil characterized by a differential scanning calorimetry thermogram substantially as shown in FIG. 2.

A sixth aspect of the present invention provides crystalline form B olmesartan medoxomil characterized by a Raman spectrum substantially as shown in FIG. 4.

A seventh aspect of the present invention provides crystalline form B olmesartan medoxomil substantially free of other forms of olmesartan medoxomil. The term “substantially free of other forms” as used herein means comprising less than about 10% of other crystalline and amorphous forms of olmesartan medoxomil, preferably less than about 5%, preferably less than about 3%, preferably less than about 2%, preferably less than about 1% (as measured by XRPD or DSC).

The crystalline form B olmesartan medoxomil of the invention possesses good dissolution characteristics and good stability over the time and temperature ranges to which pharmaceutical compositions are generally subjected, both in use and in testing for regulatory approval. Thus crystalline form B olmesartan medoxomil is suitable for pharmaceutical formulation as an angiotensin type II receptor antagonist. Thus the crystalline form B olmesartan medoxomil of the present invention is suitable for use in medicine, preferably for treating or preventing an angiotensin type II receptor mediated disorder such as hypertension.

In an eighth aspect according to the invention there is provided a process for preparing crystalline form B olmesartan medoxomil, comprising the steps of:

(a) dissolving or suspending olmesartan medoxomil in one or more organic solvent(s);
(b) causing form B to precipitate from the solution or suspension obtained in step (a); and
(c) isolating the resultant solid precipitate.

In preferred embodiments, the solvent(s) is/are selected from the group comprising tetrahydrofuran (THF), acetone, dichloromethane (DCM) and mixtures thereof. Preferably the solvent(s) is/are HPLC-grade.

In preferred embodiments, in step (a) olmesartan medoxomil is dissolved in one or more organic solvent(s). Preferably the solution or suspension obtained in step (a) is subjected to sonication or heating (preferably sonication) to aid the dissolution of the olmesartan medoxomil.

In preferred embodiments of the process, the solution or suspension obtained in step (a) is filtered, preferably through a filter having a pore size of about 0.3-1.0 μm, preferably the pore size is between about 0.4-0.6 μm, and more preferably the pore size is about 0.45 μm.

In preferred embodiments, an anti-solvent is added to the solution or suspension obtained in step (a) to cause form B to precipitate. Preferably, the anti-solvent is capable of dissolving in the solvent used in step (a). Preferably, the anti-solvent is a liquid. In a preferred embodiment, the anti-solvent is selected from the group comprising water and cyclohexane. In particularly preferred embodiments, the anti-solvent is cyclohexane, when the solvent is acetone, THF, DCM or a mixture thereof. Alternatively, the anti-solvent is water, when the solvent is THF.

In a ninth aspect according to the invention, a pharmaceutical composition is provided comprising a therapeutically or prophylactically effective amount of crystalline form B olmesartan medoxomil according to all aspects and embodiments of the invention and at least one pharmaceutically acceptable excipient.

A tenth aspect provides a method of treating or preventing an angiotensin type II receptor mediated disorder, comprising administering to a subject in need of such treatment or prevention, a therapeutically or prophylactically effective amount of crystalline form B olmesartan medoxomil according to the invention. In a particularly preferred embodiment of the invention, the disorder is hypertension.

An eleventh aspect provides a use of crystalline form B olmesartan medoxomil according to the invention in the manufacture of a medicament for the treatment or prevention of an angiotensin type II receptor mediated disorder. In a particularly preferred embodiment of the invention, the disorder is hypertension.

### BRIEF DESCRIPTION OF THE FIGURES

FIG. 1: X-Ray Powder Diffraction (XRPD) pattern of olmesartan medoxomil crystalline form B;

FIG. 2: Differential Scanning Calorimetry (DSC) heating trace of olmesartan medoxomil crystalline form B;

FIG. 3: Thermo-Gravimetric Analysis (TGA) heating trace of olmesartan medoxomil crystalline form B;

FIG. 4: Raman spectrum of olmesartan medoxomil crystalline form B.

### DETAILED DESCRIPTION OF THE INVENTION

The present invention relates to crystalline form B olmesartan medoxomil. Such a compound has not previously been described in the prior art.

The crystalline form B olmesartan medoxomil of the invention may be prepared in one embodiment by dissolving olmesartan medoxomil in an organic solvent. It has been found by the inventors that preferably the organic solvent is THF, acetone or DCM. Of course, it will be understood that a number of further organic solvents may be utilised.

In a further embodiment of the process, the olmesartan medoxomil is completely dissolved. This can be achieved by any means known in the art, but particularly preferred is exposing the solution to ultrasonication. Further embodiments comprise sonicating the solution at room temperature, which the skilled person would assume to be about 20-25° C., of course minor adjustments above or below this range are incorporated in the scope of this embodiment. In a further embodiment, the sonication is continued until a clear solution is obtained indicating that all the olmesartan medoxomil has dissolved, preferably this lasts for about 5 minutes. Alternatively, dissolution could be facilitated by heating the solution.

Further embodiments of the process comprise filtering the solution to remove any particulate matter. Such matter may act as seeds and promote the formation of unwanted crystalline forms of olmesartan medoxomil in the solution. Preferably, the solution is filtered through a filter which preferably has a pore size of between 0.1 and 1 μm. A 0.45 μm filter is particularly preferred.

Once the solution has been prepared as described above, the precipitation of the form B is facilitated. In preferred embodiments, an anti-solvent is added to the solution of olmesartan medoxomil and organic solvent. An anti-solvent can cause the precipitation of a solute. Preferably an anti-solvent is an organic or inorganic liquid. An anti-solvent is selected on the basis that it can dissolve in the solvent. It is this dissolution that provides the anti-solvent nature. Of course, it will be understood by the skilled person that the dissolution between solvent and anti-solvent may be affected by certain conditions such as temperature and pH. It is considered within the skillset of the practising artisan to determine the conditions best suited to promote adequate dissolution between the chosen solvent and anti-solvent. Preferably, the anti-solvent is selected from the group comprising water and cyclohexane. In particularly preferred embodiments, the anti-solvent is cyclohexane, when the solvent is acetone, THF, DCM or a mixture thereof. Alternatively, the anti-solvent is water, when the solvent is THF.

The precipitated solid is then isolated by any means known in the art. In particularly preferred embodiments, the solid is filtered.

The filtered solid is then allowed to dry. In certain embodiments, this may be achieved by heating, preferably by gentle heating to prevent degradation of the crystalline form B according to the invention at temperatures above about 200° C. Alternatively, the precipitate may be dried by vacuum drying. In particularly preferred embodiments, the precipitate is allowed to dry at ambient conditions, preferably in a fume-hood. Once dried, a crystalline powder is obtained.

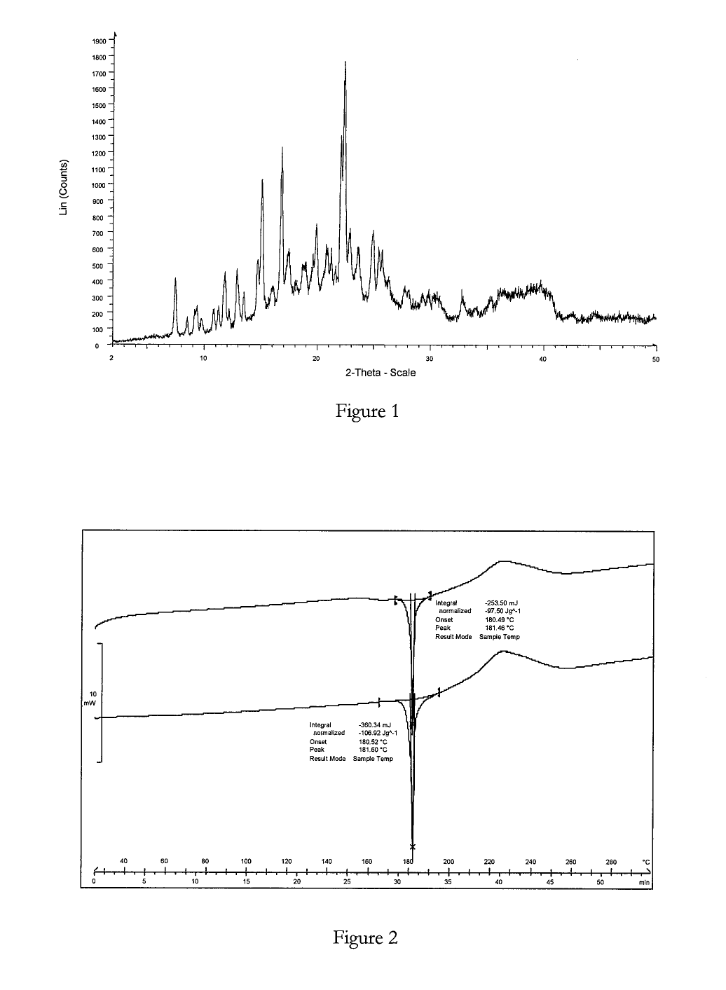

Analysis of the powder by XRPD techniques resulted in the trace shown in FIG. 1. It is important to note that the increased background in the range 35-40 °2θ is due to the XRPD sample holder.

The X-ray powder diffraction data was obtained by methods known in the art using a Bruker D8 Advance Powder Diffractometer with scintillation detector under the following parameters:

Reflection mode

Cu K\acute{α} radiation (1.5406 Å)

Scanning range: 2-50 °2θ

Step size: 0.02 °2θ

Time per step: 2 s

The powder obtained by the process according to the invention as described above and in the following examples was also subjected to Differential Scanning Calorimetry (DSC). The resulting trace is shown in FIG. 2.

The DSC thermal analysis data was obtained using a Mettler-Toledo DSC821e apparatus under the following parameters:
Temperature profile: 25-300° C. @ 5° C./min
Nitrogen purge gas, 50 ml/min
Aluminium pan, 40 ml, pierced prior to scan

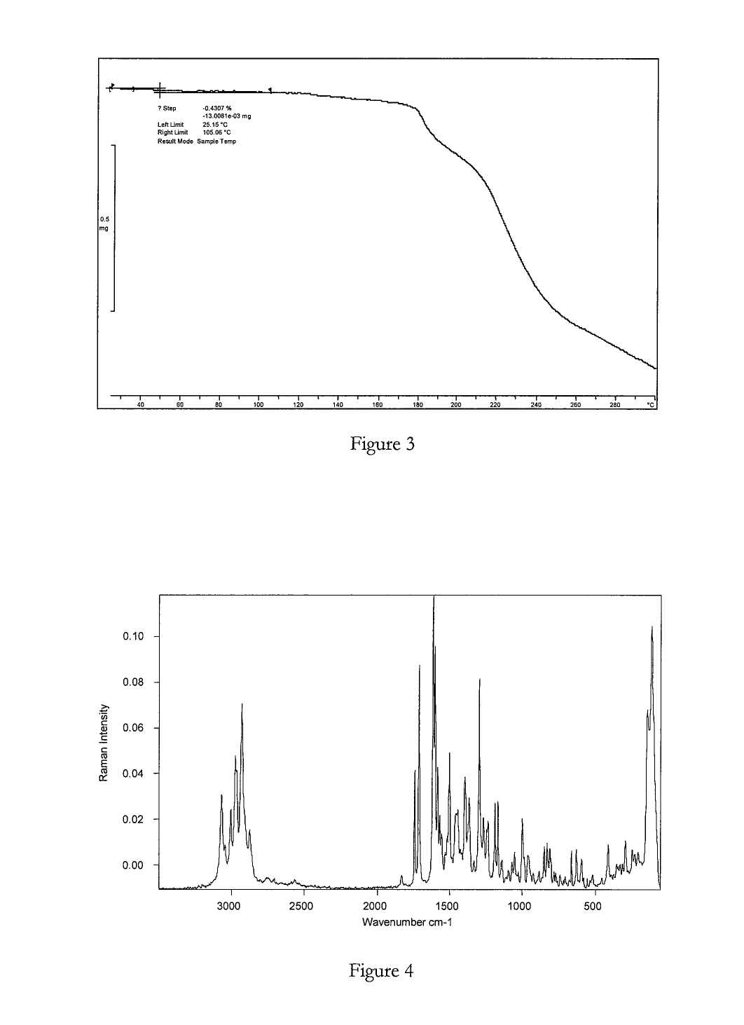

The powder obtained by the process according to the invention as described above and in the following examples was also subjected to Thermo-Gravimetric Analysis (TGA). An exemplary TGA trace is shown in FIG. 3. It can be seen that the form B according to the invention is chemically stable at processing temperatures and storage temperatures, i.e. 85362865.1 degradation by conversion to other polymorphic forms was not seen. Indeed, the DSC and XRPD experiments indicate that no polymorphic transition of crystalline form B occurs up to temperatures of ca. 170° C.

The TGA analysis data was obtained using a Mettler-Toledo TGA851e apparatus under the following parameters:

Temperature profile: 25-300° C. @ 5° C./min

Nitrogen purge gas, 50 ml/min

Aluminium pan, 40 ml, pierced prior to scan

Analysis of the obtained powder by Raman spectroscopy resulted in the spectrum shown in FIG. 4.

The Raman spectrum was obtained by methods known in the art using a Bruker Equinox 55 spectrometer with a RFS100 attachment.

Nd:YAG laser 1064 nm, 500 mW focused

b 3500-50 cm-1, resolution: 2 cm-1

64 scans

Comparison of Raman spectra of novel crystalline form B with prior art form A shows that the following Raman bands are most characteristic of form B (in comparison with prior art form A):

ca. 1580-1585 cm-1:
slightly higher wavenumber and stronger intensity than form A band

ca. 1550-1560 cm-1:
weaker intensity than form A band

ca. 1330-1335 cm-1:
slightly higher wavenumber and weaker intensity than form A band

ca. 1135-1145 cm-1:
slightly higher wavenumber and stronger intensity than form A band

ca. 950-970 cm-1:
broad band with pronounced intensity>960 cm-1 as opposed to weak shoulder>960 cm-1 in form A spectrum

ca. 510-530 cm-1:
slightly higher wavenumber and pronounced tailing>525 cm-1 compared to form A band, weaker intensity than form A band

ca. 320-360 cm-1:
evenly distributed medium intensity pattern over three absorption bands, as opposed to high intensity at ca. 330 cm-1, low intensity at ca. 345 cm-1, and medium intensity at ca. 355 cm-1 in form A spectrum

ca. 290-300 cm-1:
slightly higher wavenumber than form A band

ca. 80-160 cm-1:
weaker intensity at ca. 110 cm-1+ more pronounced shoulder at ca. 100 cm-1+ no clear minimum at ca. 130 cm-1 compared to form A spectrum

Illustrative of the invention is a pharmaceutical composition made by mixing crystalline form B olmesartan medoxomil according to the invention and a pharmaceutically acceptable carrier. A further embodiment of the invention is a process for making a pharmaceutical composition comprising mixing crystalline form B olmesartan medoxomil according to the invention and a pharmaceutically acceptable carrier. An example of the invention is a method for the treatment of an angiotensin type II receptor mediated disorder in a subject in need thereof, comprising administering to the subject a therapeutically effective amount of crystalline form B olmesartan medoxomil according to any of the embodiments of the invention or pharmaceutical compositions described above. Also included in the invention is the use of crystalline form B olmesartan medoxomil, which in preferred embodiments is substantially free of other forms of olmesartan medoxomil, for the preparation of a medicament for treating an angiotensin type II receptor mediated disorder in a subject in need thereof.

Pharmaceutical compositions of the present invention contain crystalline form B olmesartan medoxomil. It is preferred that the crystalline form B olmesartan medoxomil is substantially pure, but this is non-limiting to the working of the invention. The crystalline form B olmesartan medoxomil prepared by the processes of the present invention is ideal for formulation of pharmaceutical products. In addition to the active ingredient(s), the pharmaceutical compositions of the present invention may contain one or more excipients. Excipients are added to the composition for a variety of purposes. Diluents increase the bulk of a solid pharmaceutical composition and may make a pharmaceutical dosage form containing the composition easier for the patient and care giver to handle. Diluents for solid compositions include, for example, microcrystalline cellulose (e.g. Avicel®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit®), potassium chloride, powdered cellulose, sodium chloride, sorbitol and talc.

Solid pharmaceutical compositions that are compacted into a dosage form, such as a tablet, may include excipients whose functions include helping to bind the active ingredient and other excipients together after compression. Binders for solid pharmaceutical compositions include acacia, alginic acid, carbomer (e.g. Carbopol®), carboxymethyl cellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminium silicate, maltodextrin, methyl cellulose, polymethacrylates, povidone (e.g. Kollidon®, Plasdone®), pregelatinized starch, sodium alginate and starch.

The dissolution rate of a compacted solid pharmaceutical composition in the patient's stomach may be increased by the addition of a disintegrant to the composition. Disintegrants include alginic acid, carboxymethyl cellulose calcium, carboxymethyl cellulose sodium (e.g. Ac-Di-Sol®, Primellose®), colloidal silicon dioxide, croscarmellose sodium, crospovidone (e.g. Kollidon®, Polyplasdone®), guar gum, magnesium aluminium silicate, methyl cellulose, microcrystalline cellulose, polacrilin potassium, powdered cellulose, pregelatinized starch, sodium alginate, sodium starch glycolate (e.g. Explotab®) and starch.

Glidants can be added to improve the flowability of a non-compacted solid composition and to improve the accuracy of dosing. Excipients that may function as glidants include colloidal silicon dioxide, magnesium trisilicate, powdered cellulose, starch, talc and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc and zinc stearate.

Flavouring agents and flavour enhancers make the dosage form more palatable to the patient. Common flavouring agents and flavour enhancers for pharmaceutical products that may be included in the composition of the present invention include maltol, vanillin, ethyl vanillin, menthol, citric acid, fumaric acid, ethyl maltol and tartaric acid.

Solid and liquid compositions may also be dyed using any pharmaceutically acceptable colourant to improve their appearance and/or facilitate patient identification of the product and unit dosage level.

In liquid pharmaceutical compositions of the present invention, olmesartan medoxomil and any other solid excipients are dissolved or suspended in a liquid carrier such as water, vegetable oil, alcohol, polyethylene glycol, propylene glycol or glycerin.

Liquid pharmaceutical compositions may further contain emulsifying agents to disperse uniformly throughout the composition an active ingredient or other excipient that is not soluble in the liquid carrier. Emulsifying agents that may be useful in liquid compositions of the present invention include, for example, gelatin, egg yolk, casein, cholesterol, acacia, tragacanth, chondrus, pectin, methyl cellulose, carbomer, cetostearyl alcohol and cetyl alcohol.

Liquid pharmaceutical compositions of the present invention may also contain a viscosity enhancing agent to improve the mouth-feel or organoleptic qualities of the product and/or coat the lining of the gastrointestinal tract. Such agents include acacia, alginic acid, bentonite, carbomer, carboxymethyl cellulose calcium or sodium, cetostearyl alcohol, methyl cellulose, ethyl cellulose, gelatin, guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth and xanthan gum.

Sweetening agents such as sorbitol, saccharin, sodium saccharin, sucrose, aspartame, fructose, mannitol and invert sugar may be added to improve the taste.

Preservatives and chelating agents such as alcohol, sodium benzoate, butylated hydroxytoluene, butylated hydroxyanisole and ethylenediaminetetraacetic acid may be added at levels safe for ingestion to improve storage stability.

According to the present invention, a liquid composition may also contain a buffer such as gluconic acid, lactic acid, citric acid or acetic acid, sodium gluconate, sodium lactate, sodium citrate or sodium acetate.

Selection of excipients and the amounts used may be readily determined by the formulation scientist based upon experience and consideration of standard procedures and reference works in the field.

The solid compositions of the present invention include powders, granulates, aggregates and compacted compositions. The dosages include dosages suitable for oral, buccal, rectal, parenteral (including subcutaneous, intramuscular, and intravenous), inhalant and ophthalmic administration. Although the most suitable administration in any given case will depend on the nature and severity of the condition being treated, the most preferred route of the present invention is oral. The dosages may be conveniently presented in unit dosage form and prepared by any of the methods well known in the pharmaceutical arts. Dosage forms include solid dosage forms like tablets, powders, capsules, suppositories, sachets, troches and lozenges, as well as liquid syrups, suspensions and elixirs.

The dosage form of the present invention may be a capsule containing the composition, preferably a powdered or granulated solid composition of the invention, within either a hard or soft shell. The shell may be made from gelatin and optionally contain a plasticizer such as glycerin and sorbitol, and an opacifying agent or colourant. The active ingredient and excipients may be formulated into compositions and dosage forms according to methods known in the art.

A composition for tableting or capsule filling may be prepared by wet granulation. In wet granulation, some or all of the active ingredient and excipients in powder form are blended and then further mixed in the presence of a liquid, typically water, that causes the powders to clump into granules. The granulate is screened and/or milled, dried and then screened and/or milled to the desired particle size. The granulate may then be tableted, or other excipients may be added prior to tableting, such as a glidant and/or a lubricant.

A tableting composition may be prepared conventionally by dry blending. For example, the blended composition of the active and excipients may be compacted into a slug or a sheet and then comminuted into compacted granules. The compacted granules may subsequently be compressed into a tablet.

As an alternative to dry granulation, a blended composition may be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate and colloidal silica. The proper use of these and other excipients in direct compression tableting is known to those in the art with experience and skill in particular formulation challenges of direct compression tableting.

A capsule filling of the present invention may comprise any of the aforementioned blends and granulates that were described with reference to tableting, however, they are not subjected to a final tableting step.

The following paragraphs enumerated consecutively from 1 through 28 provide for various aspects of the present invention. In one embodiment, the present invention provides:

1. Crystalline form B olmesartan medoxomil.
2. Crystalline form B olmesartan medoxomil having at least one of the characteristic XRPD peaks with 2θ values at 9.7°, 12.1°, 13.5°, 15.1°, 17.4°, 18.1°, and 21.2°±0.2°.
3. Crystalline form B olmesartan medoxomil having at least five of the characteristic XRPD peaks with 2θ values and d values at about:

| Angle | d value |
|---|---|
| 2-Theta ° | Angstrom |
| 7.4 | 11.89 |
| 8.5 | 10.44 |
| 9.2 | 9.62 |
| 9.7 | 9.08 |
| 10.8 | 8.20 |
| 11.2 | 7.89 |
| 11.8 | 7.51 |
| 12.1 | 7.28 |
| 12.9 | 6.88 |
| 13.5 | 6.56 |
| 14.7 | 6.02 |
| 15.1 | 5.88 |
| 16.0 | 5.54 |
| 16.8 | 5.28 |
| 17.4 | 5.08 |
| 18.1 | 4.91 |
| 18.8 | 4.72 |
| 19.6 | 4.53 |
| 19.9 | 4.47 |
| 20.8 | 4.26 |
| 21.2 | 4.19 |
| 21.5 | 4.12 |
| 22.1 | 4.03 |
| 22.3 | 3.98 |
| 22.8 | 3.89 |
| 23.6 | 3.77 |
| 24.3 | 3.66 |
| 24.9 | 3.57 |
| 25.4 | 3.50 |
| 25.7 | 3.46 |
| 26.3 | 3.38 |
| 27.7 | 3.21 |
| 28.1 | 3.17 |
| 29.3 | 3.04 |
| 29.8 | 3.00 |

4. Crystalline form B olmesartan medoxomil characterized by an X-ray powder diffraction pattern substantially as shown in FIG. 1.
5. Crystalline form B olmesartan medoxomil characterized by a differential scanning calorimetry thermogram with an endothermic peak at about 181° C.
6. Crystalline form B olmesartan medoxomil characterized by a differential scanning calorimetry thermogram substantially as shown in FIG. 2.
7. Crystalline form B olmesartan medoxomil characterized by a Raman spectrum substantially as shown in FIG. 4.
8. Crystalline form B olmesartan medoxomil substantially free of other forms of olmesartan medoxomil.
9. Crystalline form B olmesartan medoxomil according to any one of paragraphs 1 to 8, for use in medicine.
10. Crystalline form B olmesartan medoxomil according to any one of paragraphs 1 to 9, for treating or preventing an angiotensin type II receptor mediated disorder.
11. Crystalline form B olmesartan medoxomil according to any one of paragraphs 1 to 10, for treating or preventing hypertension.
12. A process for preparing crystalline form B olmesartan medoxomil, comprising the steps of:
(a) dissolving or suspending olmesartan medoxomil in one or more organic solvent(s);
(b) causing form B to precipitate from the solution or suspension obtained in step (a); and
(c) isolating the resultant solid precipitate.
13. A process according to paragraph 12, wherein the solvent(s) is/are tetrahydrofuran (THF), acetone, dichloromethane (DCM) or a mixture thereof.
14. A process according to paragraph 12 or 13, wherein the solution or suspension obtained in step (a) is subjected to sonication or heating to aid the dissolution of the olmesartan medoxomil.
15. A process according to any one of paragraphs 12 to 14, wherein the solution or suspension obtained in step (a) is filtered.
16. A process according to paragraph 15, wherein the solution or suspension is filtered through a filter having a pore size of 0.3-1.0 μm.
17. A process according to paragraph 16, wherein the solution or suspension is filtered through a filter having a pore size of 0.4-0.6 μm.
18. A process according to paragraph 17, wherein the solution or suspension is filtered through a filter having a pore size of about 0.45 p.m.
19. A process according to any one of paragraphs 12 to 18, wherein an anti-solvent is added to the solution obtained in step (a) to cause form B to precipitate.
20. A process according to paragraph 19, wherein the anti-solvent is water or cyclohexane.
21. A process according to paragraph 20, wherein the anti-solvent is cyclohexane when the solvent is acetone, THF, DCM or a mixture thereof.
22. A process according to paragraph 20, wherein the anti-solvent is water when the solvent is THF.
23. A process according to any one of paragraphs 12 to 22, wherein the solvent(s) is/are HPLC-grade.
24. A pharmaceutical composition comprising crystalline form B olmesartan medoxomil according to any one of paragraphs 1 to 11, or prepared by a process according to any one of paragraphs 12 to 23, and at least one pharmaceutically acceptable excipient.
25. A method of treating or preventing an angiotensin type II receptor mediated disorder, comprising administering to a subject in need of such treatment or prevention, a therapeutically or prophylactically effective amount of crystalline form B olmesartan medoxomil according to any one of paragraphs 1 to 11, crystalline form B olmesartan medoxomil prepared by a process according to any one of paragraphs 12 to 23, or a composition according to paragraph 24.
26. A method according to paragraph 25, wherein the disorder is hypertension.
27. Use of crystalline form B olmesartan medoxomil according to any one of paragraphs 1 to 11, use of crystalline form B olmesartan medoxomil prepared by a process according to any one of paragraphs 12 to 23, or use of a composition according to paragraph 24, in the manufacture of a medicament for the treatment or prevention of an angiotensin type II receptor mediated disorder.
28. A use according to paragraph 27, wherein the disorder is hypertension.

The invention is illustrated in more detail by the following non-limiting examples.

### EXAMPLES

Examples 1-4 show processes to obtain novel crystalline form B of olmesartan medoxomil. The olmesartan medoxomil obtained was characterized by X-Ray Powder Diffraction, DSC, TGA and Raman spectroscopy, and found to be crystalline form B having traces typified in FIGS. 1-4.

### Example 1

### Precipitation of Olmesartan Medoxomil Crystalline Form B by Use of Water as Anti-Solvent from THF Solutions of Olmesartan Medoxomil

### Example 1a

Ca. 200 mg olmesartan medoxomil was dissolved in ca. 20 ml HPLC-grade THF. To achieve complete dissolution, the solution was exposed to ultrasonication for 5 minutes at room temperature (25° C.). The resulting clear solution was filtered via a 0.45 pin filter, and agitated on a magnetic stirrer with a polytetrafluoroethylene (PTFE)-coated magnetic stirring rod inserted into the THF solution. 80 ml of de-ionised (DI) water were gradually dropped into the heavily agitated THF solution, yielding a fine white precipitate after addition of ca. 40 ml DI-water. The precipitate was filtered off and the wet filter cake was allowed to dry at ambient conditions for several hours. A dry fine white powder was obtained.

### Example 1b

Ca. 200 mg olmesartan medoxomil was dissolved in ca. 20 ml HPLC-grade THF. To achieve complete dissolution, the solution was exposed to ultrasonication for 5 minutes at room temperature (25° C.). The resulting clear solution was filtered via a 0.45 μm filter, and afterwards gradually dropped into a heavily agitated DI-water reservoir (80 ml), which was agitated on a magnetic stirrer with a PTFE-coated magnetic stirring rod inserted into the DI-water reservoir. A fine white precipitate was obtained, which was filtered off, and the wet filter cake was allowed to dry at ambient conditions for several hours. A dry fine white powder was obtained.

### Example 2

### Precipitation of Olmesartan Medoxomil Crystalline Form B by Use of Cyclohexane as Anti-Solvent from Acetone Solutions of Olmesartan Medoxomil

### Example 2a

Ca. 200 mg olmesartan medoxomil was added to ca. 20 ml HPLC-grade acetone. To achieve complete dissolution, the dispersion was exposed to ultrasonication for 5 minutes at room temperature (25° C.). The resulting thin dispersion was filtered via a 1 μm filter, and agitated on a magnetic stirrer with a PTFE-coated magnetic stirring rod inserted into the acetone solution. 260 ml of HPLC-grade cyclohexane were gradually dropped into the heavily agitated acetone solution, yielding a fine white precipitate after addition of ca. 200 ml cyclohexane. The precipitate was filtered off (Whatman filter paper, no. 1) and the wet filter cake was allowed to dry at ambient conditions in a fume-hood for several hours. A dry fine white powder was obtained, which could easily be collected from the dried filter paper.

### Example 2b

Ca. 100 mg olmesartan medoxomil was added to ca. 10 ml HPLC-grade acetone. To achieve complete dissolution, the dispersion was exposed to ultrasonication for 5 minutes at room temperature (25° C.). The resulting thin dispersion was filtered via a 1 μm filter, and afterwards gradually dropped into a heavily agitated HPLC-grade cyclohexane reservoir (150 ml), which was agitated on a magnetic stirrer with a PTFE-coated magnetic stirring rod inserted into the cyclohexane reservoir. A fine white precipitate was obtained, which was filtered off, and the wet filter cake was allowed to dry at ambient conditions for several hours. A dry fine white powder was obtained.

### Example 3

### Precipitation of Olmesartan Medoxomil Crystalline Form B by Use of Cyclohexane as Anti-Solvent from THF Solutions of Olmesartan Medoxomil

### Example 3a

Ca. 200 mg olmesartan medoxomil was dissolved in ca. 20 ml HPLC-grade THF. To achieve complete dissolution, the solution was exposed to ultrasonication for 5 minutes at room temperature (25° C.). The resulting clear solution was filtered via a 0.45 μm filter, and agitated on a magnetic stirrer with a PTFE-coated magnetic stirring rod inserted into the THF solution. 150 ml of HPLC-grade cyclohexane were gradually dropped into the heavily agitated THF solution, yielding a fine white precipitate after addition of ca. 100 ml cyclohexane. The precipitate was filtered off and the wet filter cake was allowed to dry at ambient conditions for several hours. A dry fine white powder was obtained.

### Example 3b

Ca. 200 mg olmesartan medoxomil was dissolved in ca. 20 ml HPLC-grade THF. To achieve complete dissolution, the solution was exposed to ultrasonication for 5 minutes at room temperature (25° C.). The resulting clear solution was filtered via a 0.45 μm filter, and afterwards gradually dropped into a heavily agitated HPLC-grade cyclohexane reservoir (150 ml), which was agitated on a magnetic stirrer with a PTFE-coated magnetic stirring rod inserted into the cyclohexane reservoir. A fine white precipitate was obtained, which was filtered off, and the wet filter cake was allowed to dry at ambient conditions for several hours. A dry fine white powder was obtained, which could easily be collected from the dried filter paper.

### Example 4

### Precipitation of Olmesartan Medoxomil Crystalline Form B by Use of Cyclohexane as Anti-Solvent from a Dichloromethane Solution of Olmesartan Medoxomil

Ca. 200 mg olmesartan medoxomil was added to ca. 20 ml HPLC-grade dichloromethane. To achieve complete dissolution, the dispersion was exposed to ultrasonication for 5 minutes at room temperature (25° C.). The resulting thin dispersion was filtered via a 0.45 μm filter, and agitated on a magnetic stirrer with a PTFE-coated magnetic stirring rod inserted into the dichloromethane solution. 100 ml of HPLC-grade cyclohexane were gradually dropped into the heavily agitated dichloromethane solution, yielding a fine white precipitate after addition of ca. 50 ml cyclohexane. The precipitate was filtered off (Whatman filter paper, no. 5) and the wet filter cake was allowed to dry at ambient conditions in a fume-hood for several hours. A dry fine white powder was obtained, which could easily be collected from the dried filter paper.

What is claimed is:

1. Crystalline form B olmesartan medoxomil.

2. Crystalline form B olmesartan medoxomil according to claim 1, having at least one of the characteristic XRPD peaks with 2θ values at 9.7°, 12.1°, 13.5°, 15.1°, 17.4°, 18.1°, and 21.2°±0.2°.

3. Crystalline form B olmesartan medoxomil according to claim 1, having at least five of the characteristic XRPD peaks with 2θ values and d values at about:

| Angle | d value |
|---|---|
| 2-Theta ° | Angstrom |
| 7.4 | 11.89 |
| 8.5 | 10.44 |
| 9.2 | 9.62 |
| 9.7 | 9.08 |
| 10.8 | 8.20 |
| 11.2 | 7.89 |
| 11.8 | 7.51 |
| 12.1 | 7.28 |
| 12.9 | 6.88 |
| 13.5 | 6.56 |
| 14.7 | 6.02 |
| 15.1 | 5.88 |
| 16.0 | 5.54 |
| 16.8 | 5.28 |
| 17.4 | 5.08 |
| 18.1 | 4.91 |
| 18.8 | 4.72 |
| 19.6 | 4.53 |
| 19.9 | 4.47 |
| 20.8 | 4.26 |
| 21.2 | 4.19 |
| 21.5 | 4.12 |
| 22.1 | 4.03 |
| 22.3 | 3.98 |
| 22.8 | 3.89 |
| 23.6 | 3.77 |
| 24.3 | 3.66 |
| 24.9 | 3.57 |
| 25.4 | 3.50 |
| 25.7 | 3.46 |
| 26.3 | 3.38 |
| 27.7 | 3.21 |
| 28.1 | 3.17 |
| 29.3 | 3.04 |
| 29.8 | 3.00 |

4. Crystalline form B olmesartan medoxomil according to claim 1, characterized by an X-ray powder diffraction pattern substantially as shown in FIG. 1.

5. Crystalline form B olmesartan medoxomil according to claim 1, characterized by a differential scanning calorimetry thermogram with an endothermic peak at about 181° C.

6. Crystalline form B olmesartan medoxomil according to claim 1, characterized by a differential scanning calorimetry thermogram substantially as shown in FIG. 2.

7. Crystalline form B olmesartan medoxomil according to claim 1, characterized by a Raman spectrum substantially as shown in FIG. 4.

8. Crystalline form B olmesartan medoxomil according to claim 1, substantially free of other forms of olmesartan medoxomil.

9. Crystalline form B olmesartan medoxomil according to claim 1, for:
(i) use in medicine; and/or
(ii) treating or preventing an angiotensin type II receptor mediated disorder; and/or
(iii) treating or preventing hypertension.

10. A process for preparing crystalline form B olmesartan medoxomil, comprising the steps of:
(a) dissolving or suspending olmesartan medoxomil in one or more organic solvent(s);
(b) causing form B to precipitate from the solution or suspension obtained in step (a); and
(c) isolating the resultant solid precipitate.

11. A process according to claim 10, wherein the solvent(s) is/are tetrahydrofuran (THF), acetone, dichloromethane (DCM) or a mixture thereof.

12. A process according to claim 10, wherein the solution or suspension obtained in step (a) is subjected to sonication or heating to aid the dissolution of the olmesartan medoxomil.

13. A process according to claim 10, wherein:
(i) the solution or suspension obtained in step (a) is filtered; and/or
(ii) the solution or suspension obtained in step (a) is filtered through a filter having a pore size of 0.3-1.0 μm or a pore size of 0.4-0.6 μm or a pore size of about 0.45 μm.

14. A process according to claim 10, wherein:
(i) an anti-solvent is added to the solution obtained in step (a) to cause form B to precipitate; and/or
(ii) an anti-solvent is added to the solution obtained in step (a) to cause form B to precipitate, and wherein the anti-solvent is water or cyclohexane; and/or
(iii) an anti-solvent is added to the solution obtained in step (a) to cause form B to precipitate, and wherein the anti-solvent is cyclohexane when the solvent is acetone, THF, DCM or a mixture thereof; and/or
(iv) an anti-solvent is added to the solution obtained in step (a) to cause form B to precipitate, and wherein the anti-solvent is water when the solvent is THF.

15. A process according to claim 10, wherein the solvent(s) is/are HPLC-grade.

16. A pharmaceutical composition comprising crystalline form B olmesartan medoxomil according to claim 1 and at least one pharmaceutically acceptable excipient.

17. A pharmaceutical composition comprising crystalline form B olmesartan medoxomil prepared by a process according to claim 10 and at least one pharmaceutically acceptable excipient.

18. A method of treating or preventing an angiotensin type II receptor mediated disorder, comprising administering to a subject in need of such treatment or prevention a therapeutically or prophylactically effective amount of crystalline form B olmesartan medoxomil according to claim 1.

19. A method according to claim 18, wherein the disorder is hypertension.

20. A method of treating or preventing an angiotensin type II receptor mediated disorder, comprising administering to a subject in need of such treatment or prevention a therapeutically or prophylactically effective amount of crystalline form B olmesartan medoxomil prepared by a process according to claim 10.

21. A method according to claim 20, wherein the disorder is hypertension.
